(19) United States
(12) Patent Application Publication
(10) Pub. No.: US 2014/0371837 A1
(43) Pub. Date: Dec. 18, 2014

(54) DRUG-ELUTING SHEATH

(71) Applicant: Jung-Tang Huang, Taipei (TW)

(72) Inventors: Jung-Tang Huang, Taipei (TW); Shen Sun, Taipei (TW)

(21) Appl. No.: 13/917,631

(22) Filed: Jun. 13, 2013

(51) Int. Cl. A61L 31/16; A61F 2/958

(52) U.S. Cl. CPC A61L 31/16; A61F 2/958
USPC 623/1.11

(57) ABSTRACT

A drug-eluting sheath (DES) comprises an inner elastic layer, an outer elastic layer, and at least a therapeutic agent layer; said therapeutic agent layer is sandwiched partly between said inner layer and said outer layer. Said outer layer has several splittable structures on its outer surface. The DES is put on a slightly flattened catheter balloon, which guarantees both the DES and the balloon are elastically combined. The balloon carried with the DES is advanced to the treatment site of the tubular organ, and then inflated, wherein the inflating splits the outer elastic layer to expose the therapeutic agent layer to the treatment site. After treatment, the balloon is deflated, withdrawn, cleaned, and replaced with a new DES for repetitively reusing the balloon to the same patient.

### TECHNOLOGY FIELD

The present invention relates to a drug-eluting sheath, which is carried by balloon catheter to cure the disease of tubular tissue, and the sheath comprises: an elastic inner layer; a therapeutic agent layer deposited on at least part of the elastic inner layer; and an elastic outer layer deposited on the elastic inner layer and the therapeutic agent layer.

### BACKGROUND OF THE INVENTION

In recent years, population aging around the world becomes more significant, occlusive vascular disease is thus in huge increase, which is especially true in atherosclerosis disease case. And the most commonly seen cure methods are:

(1) Bypass surgery: It has higher injury, larger risk and is more time-consuming; although it has better long term effect, yet it has stayed a role in the rear line therapy.

(2) Endovascular therapy: The injury is smaller, the risk is smaller, and it saves more time; the long term effect is bad, yet repeated therapy is possible, hence, it has stayed a role in the first line therapy, and it includes mainly two types (a) Balloon dilation; (b) endovascular stent.

Currently, the therapeutic process is as in the following:

(a) Inspect; next perform balloon dilation; if it retracts back, then use endovascular stent; however, if it is a failure, then perform bypass surgery.

(b) If it is 100% occlusion in femoral artery and if stent is not used but balloon catheter is used, then patency rate one year later is 20-30%; however, if bare metal stent is used, then patency rate can be 60%; if drug eluting stent is used, then the patency rate can be 80%.

(c) Even if drug eluting stent is used, stenosis still occurs, and it will become more difficult to be handled; what is more important is, about ⅔ of the cases cannot use the stent (joint could easily get broken, and stenosis could occur under the knee due to blood vessel size too small).

The current trend around the world is:

(a) Inspect; then perform balloon dilation; however, if it retracts back, then use atherectomy device drug eluting balloon catheter; if grafting occurs, then use endovascular stent; if it is a failure, then perform bypass surgery.

(b) The use of drug eluting balloon catheter can prevent from the use of stent (Stent itself will irritate the blood vessel to get narrower, and it will become more difficult to handle); drug eluting balloon can be used on the joint or the body part below the knees.

However, currently, drug eluting balloon is very expensive and can only be used for one time, and one leg with disease might take 3-5 pieces, which cost about USD7,000-12,000. If we can let the doctor, on the operating table, put the “drug-eluting sheath” containing Paclitaxel to the existed PTA balloon catheter, then balloon catheter will be able to be used repeatedly, and in each use, only new “drug-eluting sheath” needs to be replaced, hence, the ease of use can be greatly enhanced, and the financial load of the patient can then be greatly reduced.

In addition, drug eluting balloon catheter is basically to put medicine and add an outer film directly on the existed balloon catheter, hence, it is limited by the thermal budget of the existed balloon catheter, and it thus puts a limit on the subsequent drug delivery and film material used. In addition, if the drug delivery and the adding of outer film is a failure, then the existed balloon catheter will have to be rejected together too, hence, the overall yield rate will be affected, hence, a technology to manufacture them separately is needed to achieve the goal of full separation of the preparation of drug eluting and the preparation of balloon catheter, then they are united into one piece. Its clinical result is consistent with the existed drug eluting balloon catheter, but the overall yield rate is higher, and its application scope is wider too.

### BRIEF SUMMARY OF THE INVENTION

One objective of this invention is to provide a device for drug eluting, through separable manufacturing technology, full separation of manufacturing of drug-eluting sheath and manufacturing of balloon catheter is achieved, then they are combined into one piece, and the clinical result is consistent with the existed drug eluting balloon catheter, but the overall yield rate is higher, and the application scope is wider.

Another objective of this invention is to provide a sheath for eluting the medicine, and the sheath is in association with balloon catheter in real application. When balloon catheter is sent to the stenosis spot or injury spot of the tubular tissue or organ, pressure applied to balloon catheter will make it swell and will enlarge the drug-eluting sheath too, when force is exerted on the weak point or line or pattern installed on the elastic outer layer of drug-eluting sheath to generate breakage, medicine will then be eluted effectively.

Yet another objective of the present invention is to provide a sheath for eluting the medicine, and the sheath is in association with balloon catheter for real application, then balloon catheter is sent to the stenosis spot or injury spot of tubular tissue or organ for therapy, then balloon catheter can be inflated, balloon catheter/drug-eluting sheath can then be taken out of the human body, then drug-eluting sheath is taken away, and balloon catheter is cleaned, then drug-eluting sheath is put on, and therapy can then be put on the same patient.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 is illustration of one type of drug-eluting sheaths of the present invention.

FIG. 2(A)-(D) is illustration of the usage process of one type of drug-eluting sheaths of the present invention.

FIG. 3(A)-(D) is illustration of the manufacturing process of one type of drug-eluting sheaths of the present invention.

FIG. 4 is illustration of embodiment of one type of applications of the present invention to, except the blood vessel, tubular tissue or tubular organ.

FIG. 5(A)-(D) is illustration of the physical therapeutic process provided by one type of drug-eluting sheaths of the present invention.

FIG. 6(A)-(D) is illustration of the physical therapeutic process provided by one type of long effect drug-eluting sheaths that can be adhered to the inner wall of the tubular tissue of the present invention.

### DETAILED DESCRIPTION OF THE INVENTION

Unless defined otherwise, all technical and scientific terms used herein have the same meanings as is commonly understood by one of skill in the art to which this invention belongs.

As used herein, the articles “a” and “an” refer to one or more than one (i.e., at least one) of the grammatical object of the article. By way of example, “an element” means one element or more than one element.

The present invention is further illustrated by the following descriptions, which are intended for mere demonstration and explanation but not limitation of the present invention to specific forms. The present invention envisions other variations in addition to those described herein. It is believed that those skilled in the art can achieve the whole scope of the present invention based on the descriptions herein.

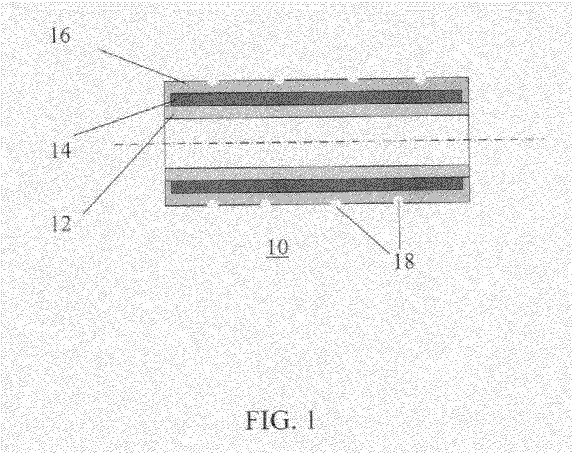

One drug-eluting sheath is provided in this invention as in FIG. 1. The drug-eluting sheath 10 mainly includes an elastic inner layer 12, an elastic outer layer 16, and at least one therapeutic agent 14 can be included in between elastic inner layer 12 and elastic outer layer 16, and elastic outer layer 16 is installed with weaker point, line or drawing 18.

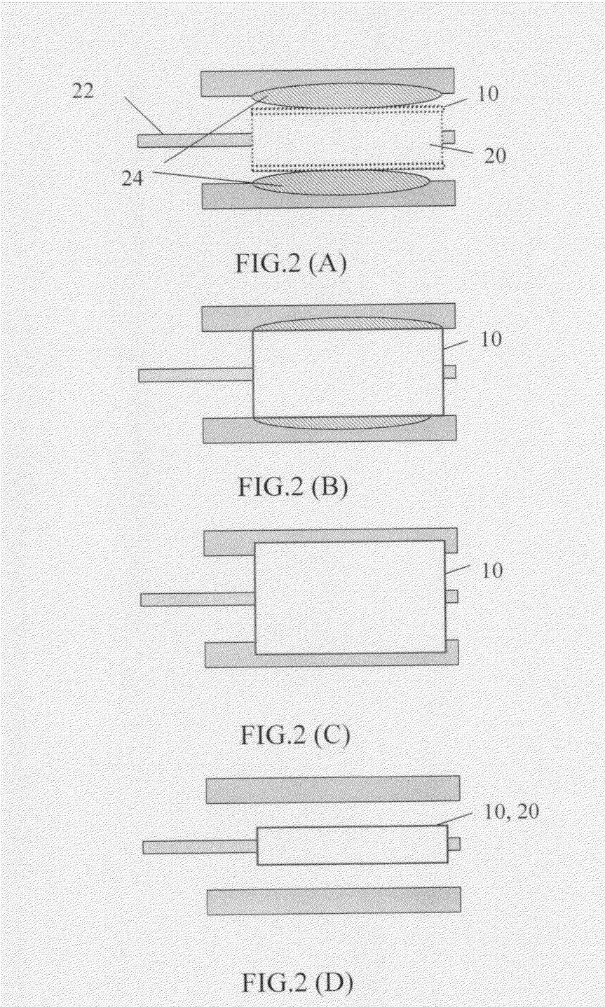

As shown in FIG. 2, in real use, when drug-eluting sheath 10 is put on slight air-filled balloon catheter 20, due to high coupling between drug-eluting sheath 10 and balloon catheter, and due to its close elasticity and compliance to balloon catheter, it can be deformed along with balloon catheter, hence, when balloon catheter runs within the blood vessel, the drug-eluting sheath will not get broken. When balloon catheter is sent to the stenosis spot or injury spot 24 of the tubular tissue, pressure exerted on balloon catheter to let it swell will enlarge the drug-eluting sheath 10 at the same time too, when force is exerted on the weak point or line or drawing of elastic outer layer 16 to let it break, therapeutic agent will then be released effectively.

Through the opening and swelling of balloon 20 itself to make external compression on drug-eluting sheath 10, it is then possible to install or coat on the inner surface of elastic inner layer of drug-eluting sheath 10 with the non-slip nano structure or non-slip material so as to keep its rough inner surface, hence, enough frictional force can be formed with the external surface of balloon 20, and it will not fall apart during the movement within the blood vessel. In addition, when the medicine is eluted, it will get separated, balloon catheter will then be withdrawn back, and drug-eluting sheath 10 will then be taken down, meanwhile, balloon catheter should be cleaned and dried, and new drug-eluting sheath 10 can be put on for the same patient for a new use. Basically, drug-eluting sheath 10 can be associated with smaller size balloon catheter so that drug-eluting sheath can be easily put on. In addition, non-slip nano structure or non-slip material can be further installed or coated on the external surface of balloon 20 so that sufficient frictional force can be formed between the inner surface of drug-eluting sheath and the outer surface of balloon 20, and no separation will then occur during the movement in the blood vessel.

Elastic inner layer 12 is a type of high temperature vulcanized silicone dispersion. Such material is material with high elongation-at-break percentage, and this value is at least 1000%.

Therapeutic agent 14 might contain for example, antineoplastic agent, antiproliferative agent, antibiotic, antithrombogenic agent, anticoagulant, antiplatelet agent, anti-inflammatory agent, or a combination of the above. For all other therapeutic agents 14, for example, fibrinolytics, therapeutic proteins or peptides, recombinant DNA product, or other bioactive agents, diagnostic agents, radioactive isotopes, or radiopaque substances, might be added into therapeutic agent layer 14 according to the expected need of the patient.

Elastic outer layer 16 is deposited above elastic inner layer 12 and therapeutic agent 14, in one embodiment, elastic outer layer 16 is one type of oxime cure silicone dispersion. Such material has lower elongation-at-break percentage than that of elastic inner layer 12, and the better value is in the range of 500-750%.

Anyone who is familiar with the prior art should know that elastic inner layer 12 and elastic outer layer 16 can also be replaced by any other bio-compatible polymer material as long as the elastomer characteristic possessed by the polymer material is the same as the above. The elastomer material for example, might be high temperature vulcanized or low temperature vulcanized silicone or its combination. In one of the embodiments, the difference between first elongation-at-break percentage and second elongation-at-break percentage should be at least 250%.

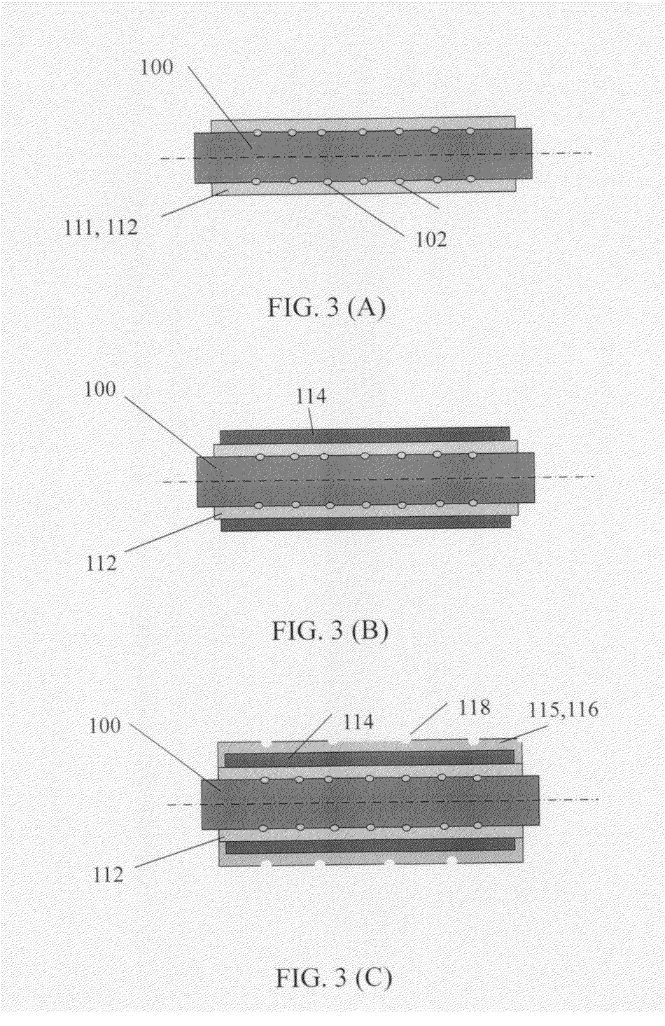

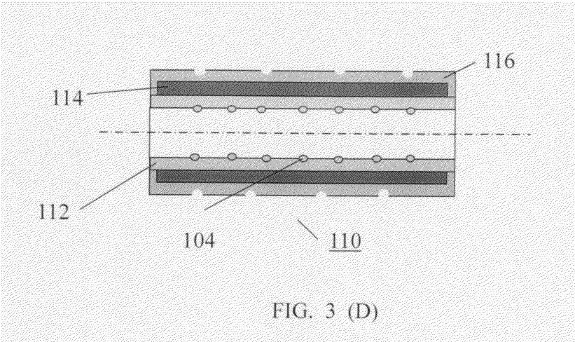

The manufacturing process of drug-eluting sheath is as shown in FIG. 3 with the following steps:

1. As shown in FIG. 3(A), a first coated layer 111 is formed on de-moldable mandrel 100; furthermore, on mandrel 100, nano to micro dented point 102 is formed, meanwhile, when the first coated layer 111 is formed, protruding point 104 can be generated to increase the frictional force.

2. Cure first coated layer 111 to form elastic inner layer 112;

3. As shown in FIG. 3 (B), a therapeutic agent layer 114 is formed on at least part of elastic inner layer 112;

4. As shown in FIG. 3 (C), a second coated layer 115 is formed above elastic inner layer 112 and therapeutic agent layer 114, and the external surface of second coated layer 115 can be illuminated by laser or UV, or accompanied with shadow mask, material weak point 118 or reduction of elongation-at-break percentage can be generated at the illuminated site;

5. Cure second coated layer 115 to form elastic outer layer 116; and

6. As shown in FIG. 3 (D), elastic inner layer/therapeutic agent layer/elastic outer layer are released from mandrel 100 to form a separable drug-eluting sheath 110;

Wherein the material of elastic inner layer has first elongation-at-break percentage, the material of elastic outer layer has second elongation-at-break percentage.

The formation of first coated layer 111 on mandrel 100 can be achieved by any prior art, for example, immersion, spraying, painting, brushing, rolling, printing and any combination. Mandrel is a mold, which has dented microstructure on the external surface so as to generate the size and dimension and the protruding microstructure in the inner surface as needed by drug-eluting sheath 110.

The liquid material needed to form first coated layer 111 basically includes elastomeric polymer. The liquid material might be solution of lactoprene or polymer in organic solution. The organic solvent might be, for example, ether, amine, ester or alcohol. In one embodiment, the liquid material means solution of silicone in xylene. In another embodiment, the liquid material means solution of silicone in hexane. Each time, mandrel is immersed in the liquid material for neutralization, then it is drawn back, then a thin film of liquid material is going to be formed on the outer surface of mandrel. The desired thickness of first coated layer can be increased through multiple immersions of mandrel.

Between each immersion, first coated layer can get partially cured so that adhesion can be easily acquired among the medium layers. The length of the curing time of the immersion layer of mandrel depends on polymer type, solvent type and the viscosity needed. Once the required thickness has been reached, first coated layer can be cured. The process better be carried out under high temperature, for example, in the range about 250° F.-350° F., for about 120 minutes-150 minutes to form elastic inner layer. In one embodiment, the curing process is platinum curing for 45 minutes at 170° F., then an additional heating is carried out at 300° F. for 135 minutes. Anyone who is familiar with the prior art will know that different combination of curing time and temperature could result in the same effect. For example, curing can be carried out under a longer time and lower temperature combination.

Next, the formation of therapeutic agent layer 114 includes the exertion of therapeutic agent onto the entire elastic inner layer 112 or any of its part. The elastic inner layer 112 part can be masked first, then mandrel is immersed in the solution to be coated, which is a process to be adapted to specific application procedure. Therapeutic agent layer can be formed through the application of any known prior art, for example, immersion, spraying, painting, brushing, rolling, printing and any combination.

After the formation of therapeutic agent layer, it can be coated above elastic inner layer and therapeutic agent layer. Mandrel coated with therapeutic agent can be immersed in outer layer polymer solution to form the second coated layer. The formation of elastic outer layer is similar to that of elastic inner layer. Elastic outer layer can also be formed through the use of prior art, for example, for example, immersion, spraying, painting, brushing, rolling, printing and any combination.

Elastic outer layer 116 and elastic inner layer 112 encloses therapeutic agent 114 between them. Through the implementation of additional immersion and drying period, second middle layer can be formed, and the thickness of second coated layer can be increased. Second middle layer can be continuously increased until the required thickness of second coated layer is reached. In order to let certain part of elastic outer layer 116 has better elasticity than that of other parts, the thickness of second coated layer might be different. Once the required thickness is reached, second coated layer can be, within room temperature range, cured from 70° F. to 77° F., and the time better be 24 hours to form elastic outer layer 116.

The above manufacturing process for drug-eluting sheath has lots of features:

1. Special mandrel 100 material can be used, for example, material that can swell and retract for several times. And above mandrel, there is nano to micro dented point 102, meanwhile, protruding point 104 can be generated while first coated layer 111 is formed so as to increase the frictional force.

2. Cure first coated layer 111 to become elastic inner layer 112. Since mandrel 100 can resist high temperature, the curing can thus be carried out at high temperature, in other words, the manufacturing efficiency and yield rate can be increased, and it facilitates the selection of more materials too, that is, the thermal budget can be greatly reduced, and it does not have to be affected by the original balloon.

3. The coating of therapeutic agent layer 114 becomes simpler.

4. On elastic outer layer 116, some mechanisms can be used to achieve weaker point or line so that in the future, when force is exerted, breakage could be easily generated. For example, after finishing the second coated layer, partial dipping of water soluble fine particle should be carried out, for example, salt powder, next, it is cured into elastic outer layer 116, then the salt powder is dissolved so that weaker point or line is partially formed in the middle of elastic outer layer 116. Or the laser or UV illumination is used, or shadow mask is used together so that the illuminated part will be material of weaker point or material with reduced elongation-at-break percentage.

Multi-Layer Medicine Sheath

For the situation where multiple medicines are needed, multiple layers can be prepared, and multiple sectional structures are also allowed. Moreover, external film of different strength can be prepared respectively, meanwhile, at different inter-layer, different medicine can be placed. In one transport process, different pressure is given to different balloon to generate different compression force so that medicine of different inter-layer will get broken according to the strength of the external film, then the medicine will be released sequentially to achieve therapeutic method similar to cocktail.

Liquid Medicine

Traditionally, drug eluting balloon is only good for the coating of solid medicine, and it usually does not include liquid medicine, hence, there is usually a limit on the medicine transport efficiency and medicine absorption. Liquid medicine usually has faster absorption and transport. The material of the outer layer and inner layer can be silicone rubber, for example DOW CORNING related series, SILASTIC MDX4-4210 Biomedical grade elastomer. Before the curing process, the medicine can be deposited or injected into the space between the outer layer and inner layer. Moreover, a notch mark can be made on the outer layer on the middle divisional part, hence, when it receives the compressional force from the balloon, breakage will be generated on the notch mark so that medicine, especially liquid medicine, can be released quickly.

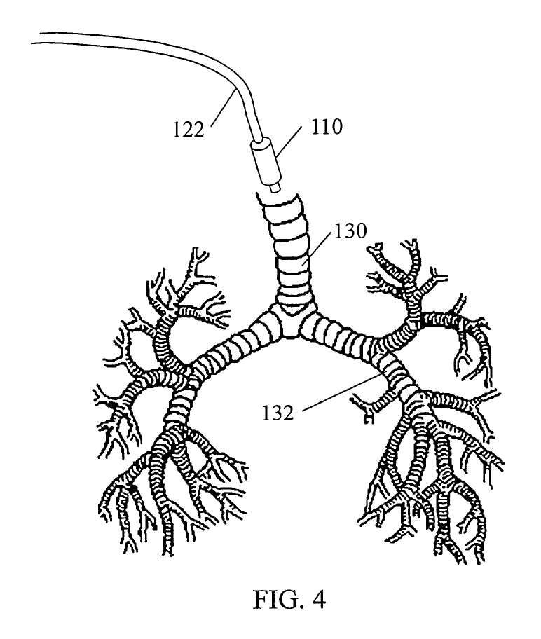

Although the above drug-eluting sheath has its application target mainly based on blood vessel stenosis, yet the application scope of the present invention is not limited to this. In addition to the blood circulation system, since there are lots of conduits in human organs, the therapy of the conduit can usually adopt the therapeutic method of balloon catheter/drug-eluting sheath. In this method, the medicine can be put very quickly, and the goal of target therapy can be achieved because the transport of medicine can be done through balloon catheter to the neighborhood of the disease spot before the medicine is released. It can be applied in narrow trachea or bronchus or inflammation or ulcer or infection as shown in FIG. 4; or the stenosis or narrowness of urinary system or urinary tract. It can also be applied in digestive system: for example, anus, rectum, small intestine, stomach, and esophagus, etc., basically, DES technology can be applied, and the entrance can be through anus or through mouth. Drug-eluting sheath can be adopted as drug delivery method for male's or female's reproductive system. It is further described as in the following:

Respiratory system, for example, trachea 130 or bronchus 132, might get narrower due to infection or the attack of malignant tumor, which is as shown in FIG. 4. In such case, drug-eluting sheath (DES) 110 of the current invention can be put on balloon catheter 122 so that medicine can be transported through balloon catheter 122 to neighborhood of the disease spot before it is expanded and medicine released, moreover, the medicine can be anti-cancer, anti-infection or anti-stenosis medicine.

In the case of occlusion or narrowing in the urinary passage of urinary system, the present invention of drug-eluting sheath (DES) can similarly be used to expand and to release anti-stenosis medicine.

If bleeding or infection is seen in the digestive system or if peptic ulcer occurs, drug-eluting sheath (DES) of the present invention can be used directly to release vasoconstrictor or anti-infection medicine on the treatment site.

For hemorrhoids bleeding or pain, drug-eluting sheath (DES) of the present invention can be used to release vasoconstrictor or pain-killing anti-inflammatory medicine.

Occlusion in canalis nasolacrimalis: Clinically, two types of persons could easily have gum in the eyes due to occlusion in canalis nasolacrimalis: One is the congenital occlusion in canalis nasolacrimalis in the new born baby, which usually is accompanied with gum in the eyes. Another case is the mucosal adhesion in the canalis nasolacrimalis for elderly people due to clinic conjunctivitis, trachoma or trauma. The drug-eluting sheath (DES) of the present invention uses canalis nasolacrimalis balloon to expand the spot in stenosis and to release the medicine, hence, it can prevent the recrudescence.

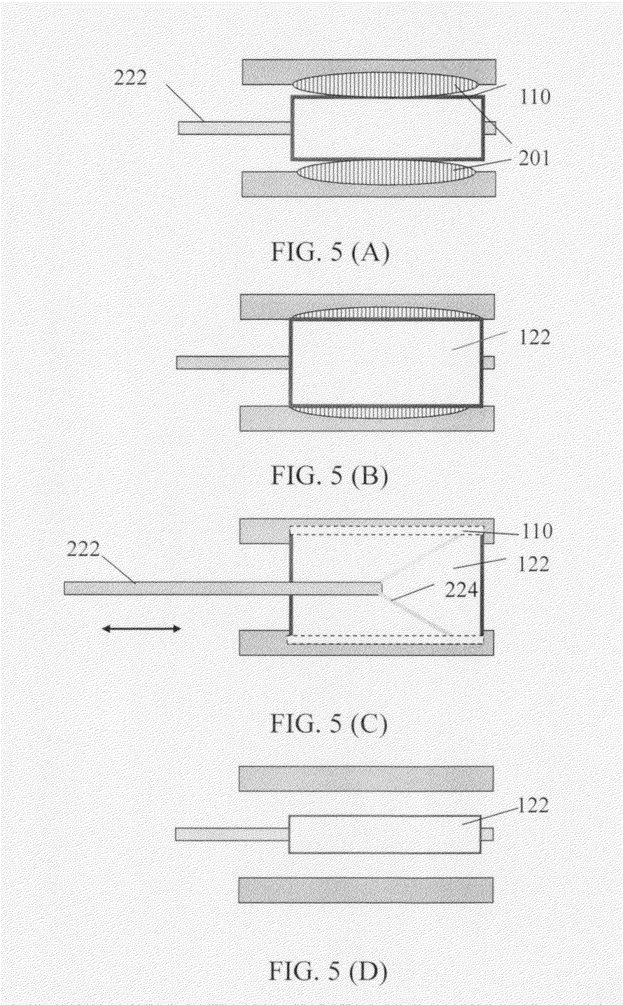

Furthermore, as shown in FIG. 5, the therapeutic agent of drug-eluting sheath (DES) of the present invention can be replaced by nano particles, for example, silica cladded with gold shell externally, or cladded with nano magnetic bead or magnetic rod, and the guiding line can be replaced by optical fiber 222. During the real application, the user can put drug-eluting sheath (DES) 110 of the present invention on the balloon catheter 122, and the transport of nano gold shell medicine can be done through balloon catheter 122 to the neighborhood tissue of the treatment site 201, for example, for example, the cancer cell as shown in FIG. 5 (A). Next, balloon catheter 122 is expanded to release drug-eluting sheath (DES) as shown in FIG. 5 (B). Next, near infrared (NIR) 224 is sent to the end point of optical fiber 222 so as to provide illumination to nano gold shell adhered to the disease spot, and since the nano gold shell has high absorption to near infrared, hence, it will generate high temperature to kill the cancer cell, which is as shown in FIG. 5(C). Finally, after a period of illumination time, the size of balloon catheter 122 can be reduced to be drawn back out of the body, which is as shown in FIG. 5 (D). Anyone who is familiar with this art should understand that nano gold shell can also be coated externally with anti-body or aptamer that has specificity to cancer cell, hence, when it is released, it can be, in its neighborhood, specifically adhered to the peripheral of the cancer cell, and it will not have any influence on the normal cell.

Long Effect Drug-Eluting Sheath

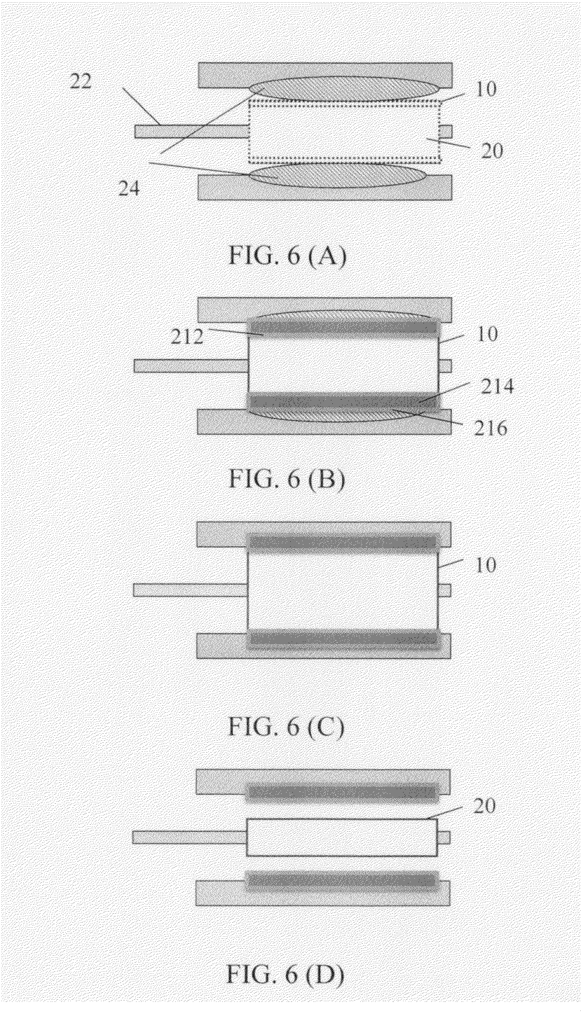

As shown in FIG. 6 (A), elastic outer layer 216 is a layer of porous or net structure, and it is bio-degradable, it can be effectively adhered to the inner wall of the blood vessel or the inner wall of the tubular organ under high pressure, or it has the inner wall of the tissue cell in stenosis, and therapeutic agent layer 214 can be preserved through the porous characteristic of elastic outer layer 216, then it will penetrate gradually into the tissue cell in stenosis. Elastic inner layer 212 is a bio-degradable material, when pressure is exerted on it, since it can be separated with balloon catheter, as shown in FIG. 6 (C), hence, the entire drug-eluting sheath can be fully adhered to the tubular inner wall and can get fully separated from balloon catheter 20, hence, balloon catheter 20 can be easily drawn back from tubular inner wall that is in stenosis or injured.

95% of the medicine of current drug eluting balloon coated on the inner wall of the blood vessel is flushed away by the blood, in this embodiment, the high adhesion characteristic of elastic outer layer to the inner wall of the blood vessel is used here, meanwhile, elastic inner layer can protect therapeutic agent layer not to be swept away by the moving blood, hence, the bio-degradable time can last for several months. The adhesive characteristic of elastic outer layer can assist the sheath to be adhered in the tissue, hence, the gradual medicine eluting at specific spot and within expected timing can be achieved. The material of elastic outer layer can be natural or artificially synthesized polymer adhesive substance. It might be natural bio-adhesive molecule containing DOPA, 3,4-dihydroxy-L-phenylalanine and catechol, or synthesized adhesive molecule of cyanoacrylate.

Balloon can be of longer structure, and drug-eluting sheaths can be several pieces in use at one time and can be put into balloon catheter at the same time. Hence, for the same tubular organ, if there are multiple ulcer or injured spots, simultaneous drug delivery mechanism can be provided, hence, multiple in and out for the replacement of balloon catheter can be avoided, and the surgical time can then be saved. In addition, there are several advantages for doing so, that is, it only needs a few lengths and dimensions for drug-eluting sheath, and if the drug delivery range is large, only multiple drug-eluting sheaths need to be serially connected.

In the above embodiment, while three-layer structure is taken as an example, however elastic outer layer and therapeutic agent can be mixed into one layer of drug-eluting bioadhesive gel, and then it forms a double-layer structural drug-eluting sheath with elastic inner layer.

Elastic outer layer can be done through press transfer printing, or together with thermal transfer printing or optical transfer printing technology, elastic outer layer can be adhered to the wet inner wall of the blood vessel or tubular organ tissue effectively and in very long term, then together with the protection from elastic inner layer, the selection of therapeutic agent can be diversified, in other words, it will not be limited to therapeutic agent that can be mixed with elastic outer layer, instead, as long as it can be enclosed by elastic inner layer and elastic outer layer. Elastic outer layer will get dilated due to force exerted on it, and its porous tissue will increase a lot after dilating, hence, therapeutic agent molecule can make effective penetration, on the contrary, the porosity of elastic inner layer is much lower or it is even non-porous elastic material, hence, even if it dilates, it will not generate pores, hence, it can prevent the therapeutic agent from diffusion into the blood, that is, through the material selection of elastic outer layer and elastic inner layer, partial fixed point drug delivery can be divided into short term and long term way, and the dosage and speed of the medicine eluting can be selected too, that is, it can be decided by the level of treatment. Meanwhile, the drug delivery can be to the inside or to the outside, for example, elastic inner layer can be made into porous material, therapeutic agent can then be eluted internally.

To sum up, the features of drug-eluting sheath are as in the followings:

Therapeutic agent can be chemical medicine, but it can also be physical nano material; moreover, it can have several dimensions, namely, diameter and length; furthermore, it has high coupling to balloon catheter, and it has elasticity and compliance close to that of balloon, meanwhile, it can deform together with balloon, and it won't get broken while it runs in the blood vessel; it will not get dissolved in the blood (if it is not under the compression of sufficient mechanical force, therapeutic agent will not get released) (Without any heating, the elastic layer of drug-eluting sheath will not get melted to release therapeutic agent); it has long term preserving characteristic; therapeutic agent can finish a release of more than 80% within very short period of time, for example, 1-10 minutes, however, it can also be used together with elastic outer layer or elastic inner layer with porous or net structure to provide long term and slow medicine eluting.

Although this invention has been disclosed as above through better embodiment, yet it is not used to limit the present invention, anyone who is familiar with the prior art can, without deviating from the spirit and scope of the present invention, make slight change and modification, and it should still fall within what is claimed of the present invention.

1. A system for treating a tubular tissue condition, the system comprising:
a balloon catheter, which is formed through the installation of a balloon on the peripheral of a tube; and
a separable and breakable drug-eluting sheath, which is put on at least part of the balloon, and the drug-eluting sheath comprises an elastic inner layer; a therapeutic agent layer deposited on at least part of the elastic inner layer; and an elastic outer layer deposited on the elastic inner layer and the therapeutic agent layer; and the elastic inner layer material has first elongation-at-break percentage, and the elastic outer layer material has second elongation-at-break percentage.

2. The system of claim 1, wherein elastic inner layer is a platinum cure silicone dispersion.

3. The system of claim 1, wherein therapeutic agent layer is selected from the group consisting of powdered medicine, fluid medicine and nano particle.

4. The system of claim 1, wherein the tubular tissue is selected from the group consisting of the blood vessel, the conduit of respiratory system, the conduit of digestive system, the conduit of urinary system or the conduit of reproductive system.

5. The system of claim 1, wherein the elastic outer layer is an oxime cure silicone dispersion.

6. The system of claim 3, wherein nano particle, through the guiding line of balloon catheter is further replaced as optical fiber, and near infrared (NIR) through the optical fiber, is illuminated on the nano particle so as to heat it up and raise its temperature.

7. A drug-eluting sheath for treating the condition of tubular tissue, the sheath comprising:
an elastic inner layer; a therapeutic agent layer deposited on at least part of the elastic inner layer; and an elastic outer layer deposited on the elastic inner layer and the therapeutic agent layer; and the elastic inner layer material has first elongation-at-break percentage, and the elastic outer layer material has second elongation-at-break percentage.

8. The drug-eluting sheath of claim 7, wherein the first elongation-at-break percentage is at least 1000%.

9. The drug-eluting sheath of claim 7, wherein the second elongation-at-break percentage is in the range from 550% to 750%.

10. The drug-eluting sheath of claim 7, wherein the difference between the first elongation-at-break percentage and the second elongation-at-break percentage should be at least 250%.

11. The drug-eluting sheath of claim 7, wherein the therapeutic agent layer is selected from the group consisting of powdered medicine, fluid medicine and nano particle.

12. The drug-eluting sheath of claim 7, wherein the tubular tissue means one of the blood vessel, the conduit of respiratory system, the conduit of digestive system, the conduit of urinary system or the conduit of reproductive system.

13. The drug-eluting sheath of claim 7, wherein the drug-eluting sheath is put on at least part of the balloon of a balloon catheter, and balloon catheter is made through the setup of a balloon on the peripheral of a tube.

14. A method to form the drug-eluting sheath as claimed in claim 7, the method comprising:
forming a first coated layer on a de-moldable mandrel;
curing the first coated layer to form the elastic inner layer;
forming the therapeutic agent layer on at least part of the elastic inner layer;
forming a second coated layer above the elastic inner layer and the therapeutic agent layer;
curing the second coated layer to form the elastic outer layer; and releasing elastic inner layer/therapeutic agent layer/elastic outer layer from the mandrel to form the drug-eluting sheath;
wherein the elastic inner layer material has first elongation-at-break percentage, and the elastic outer layer material has second elongation-at-break percentage.

15. The method of claim 14, wherein the forming of the elastic outer layer further comprises steps of, before curing the second coated layer, partial depositing fine particles on the surface of elastic outer layer, after curing, then dissolving the fine particles to forms partial weak point or line on the surface of elastic outer layer.

16. The method of claim 14, wherein the outer surface of elastic outer layer is illuminated by laser or UV, or is accompanied with shadow mask, to generate weak point or the reduction of elongation-at-break percentage on the illuminated site.

17. A method for treating a tubular tissue condition by using the drug-eluting sheath as claimed in claim 7, the method comprising:
putting the drug-eluting sheath on a part of balloon catheter, the drug-eluting sheath is a separated and breakable elastic drug delivery device comprising the elastic inner layer, the therapeutic agent layer with therapeutic effect deposited on at least part of elastic inner layer, and the elastic outer layer deposited above the elastic inner layer and the therapeutic agent layer with therapeutic effect;
advancing drug-eluting sheath along with balloon catheter to the treatment site of the tubular tissue;
inflating the balloon and the drug-eluting sheath,
wherein the weak point or line or pattern on the elastic outer layer generates breakage to release the therapeutic agent to the treatment site.

18. A drug-eluting sheath for treating condition of tubular tissue, the sheath comprising:
an elastic inner layer;
a therapeutic agent layer deposited on at least part of the elastic inner layer; and
an elastic outer layer deposited on the elastic inner layer and the therapeutic agent layer, material of the elastic outer layer is natural or artificially synthesized polymer adhesive substance;
wherein the sheath is put on at least part of the balloon of the balloon catheter, and balloon catheter is made through the installation of a balloon on the peripheral of a conduit; under the compression due to the swelling of balloon, the adhesiveness of the elastic outer layer helps the sheath the adhere to the inner wall of the blood vessel or the inner wall of other tubular tissue, consequently, therapeutic agent is eluted slowly to the tissue at specific spot and on the expected time period.

19. The drug-eluting sheath of claim 18, wherein the elastic outer layer is a porous or net structure, and bio-degradable; due to the force exerted on the sheath by inflating balloon catheter, great increase on the porous structure enhances the molecules of therapeutic agent penetrate effectively to the tissue.

20. The drug-eluting sheath of claim 18, wherein the elastic outer layer and the therapeutic agent is mixed into one layer of drug-eluting bioadhesive gel, and forms a double layer drug-eluting sheath with elastic inner layer.
